(19) United States
(12) Patent Application Publication
(10) Pub. No.: US 2019/0022340 A1
(43) Pub. Date: Jan. 24, 2019

(54) TEMPERATURE-REGULATED COLOR CHANGING QUARTZ BANGER USED FOR SMOKING MEDICINAL HERBAL CONCENTRATE

(71) Applicant: Hannifa Mayar, Uniondale, NY (US)

(72) Inventor: Hannifa Mayar, Uniondale, NY (US)

(21) Appl. No.: 15/655,875

(22) Filed: Jul. 20, 2017

(51) Int. Cl. A61M 15/06; G01K 11/12

(52) U.S. Cl. CPC A61M 15/06; A24F 5/00; G01K 11/12

(57) ABSTRACT

A water pipe attachment, specifically, a quartz banger for placing therein a smoking substance, and a color changing surface inserted therein, not limited to any location around the neck and head area of the banger to change color initially in response to heat as high as 300 to 400 degrees.

### CROSS-REFERENCE TO RELATED APPLICATION(S)

Not applicable

### BACKGROUND OF THE INVENTION

### 1. Field of Invention

The present invention relates generally to water pipe smoking devices or other filtration devices generally used for smoking cannabinoid substances or mediums, or other herbal medicines or mediums. The present invention relates particularly to attachment members to such filtration devices, and in one instance, a quartz banger (commonly called nail) used to heat concentrated herbal medicines.

### 2. Description of Related Art

The water pipe has been used for centuries to smoke tobacco, herbal substances and cannabinoid compounds. Typically, a water pipe has a bowl in which the product is placed, the bowl being mounted on top of a reservoir structure. The interior of the reservoir is partially filled with water. Extending downward from the bowl into the reservoir is a tubular hollow stem, with its lowermost extremity immersed in the water. The interior portion of the reservoir structure above the level of the water filled with ambient air forms a chamber into which smoke may be collected. Depending on the type of water pipe used, some means of drawing the ambient air and collected smoke, either one or more flexible hoses or a glass neck, extend outward from this smoke collection portion of the reservoir in fluid communication with the reservoir.

Inhaling through the means to draw out the ambient air, a smoker causes smoke to be drawn from the bowl down the stem, passing the smoke through the water in the lower portion of the reservoir. Passing upwards through the water in the form of bubbles, smoke gradually fills the smoke collection portion of the reservoir, and when sufficient smoke has been collected, passes on through the flexible hose or glass neck to the smoker. By drawing the smoke through a water reservoir, the smoke is filtered and cooled.

This basic functionality is shared by virtually all traditional water pipes, which generally differ only in size, shape, and style. Some use a lighted coal as a heat source placed in or above the bowl with the product for burning. Others may require an ignition source such as a match or butane lighter to be placed near the top of the bowl to begin burning the product.

Plant matter such as tobacco, cannabinoids, or other herbal medicines, when smoked is also not properly utilized to maximum efficiency. Consumers may heat the product to temperatures as hot as 800° to 1500° F. As much as 95% of the active material can be wasted in order to absorb 5% or less of the same. Also, much of the active ingredients, helpful medicines, and aromatics or terpenes are destroyed by combustion at this temperature. Various attempts have been tried to overcome the problems associated with smoking.

U.S. Pat. No. 8,550,091 addressed this problem by electrically heating the product and attaching a temperature sensor to the bowl to measure the temperature of smoking substance. The sensor then coordinated with a potentiometer (e.g. 110/240V) with an easily read display (analog or digital) for results. The display showed the current temperature of the substance (as sensed by the temperature sensor), and indicted with red or green lights if the temperature was safe (green safe, red not safe) for vaporizing the volatile aromatic compounds of smoking substance without undesirable combustion by-products that may harm the consumers throat or lungs. However, this product is atypical for smoking herbal concentrates. It is common practice to heat concentrates with a butane heat source instead of an electrical heat source, and more specifically, consumers of concentrates who use quartz banger attachments use butane torches. Further, temperature is harder to regulate when using a butane torch as a heat source, more so than electrical heat sources due to the direct heat application of a butane torch, and thus accurate and time-sensitive temperature indicators are imperative.

U.S. Pat. No. 6,990,978 further showed ways in which temperature could be regulated to most efficiently burn materials that release active constituents for inhalation without the creation of harmful byproducts or otherwise destroy by combustion the medicinal properties, aromatics, and active ingredients. The device related to an apparatus for vaporization that could be supplied with an information delivery and retrieval means either by USB, wireless Ethernet, and several other communicative means to permit access to an information source (e.g. a personal computer, PDA, etc.). For instance, a USB cable may be connected to the apparatus and a computer, wherein the computer delivered information to a time and temperature control means on the device. Also, information could be stored externally, such as on a web site for optimal temperatures and settings according to the substance being used. Water is not employed in the process, so the apparatus does not operate in the manner of a water pipe. In the absence of water filtration, vapor is insufficiently cooled. Additionally, this application would not serve consumers who smoke herbal concentrates. The application is purposed only for tobacco and herbal non-concentrate consumption and is heated only by electrical means. As mentioned above, this heat source is improper for consumers of herbal concentrates who use a butane torch to apply direct heat to product placed on a quartz banger.

US patent application 2007/0280652 disclosed a water pipe with a means to observe heat response by way of physical changes in the appearance of a “central vessel.” The central vessel, connected to the bowl where the product is burned, was subjected to the subsequent volatile smoke filtering through the central vessel from where the product was burned. Heat exchange between the central vessel and this source caused a change in color of glass that would be “visibly pleasing” to consumers and nothing more. The glass changed colors in response to heat for aesthetic purposes and to show that the central vessel had received heat. There was no indication on the apparatus that showed at what temperature it was operating. The color change merely indicated that the apparatus was hot without any utility beyond that. Further, the color change came as a result of color treatments of the glass. The present invention uses a single or several color changing surfaces inserted close to the neck of the banger nearest to the heating surface where the medicinal herbal concentrate will be placed for smoking. The quartz will not be treated other than by a single or multiple insertions of the color-changing surface near the neck of the banger.

### SUMMARY OF THE INVENTION

Accordingly, the present invention seeks to provide consumers with a means to accurately tell at what temperature their water pipe apparatus is operating without sacrificing the active ingredients, helpful medicines, and aromatics or terpenes that may be destroyed by combustion at high temperatures, and without producing undesirable combustion by-products that may harm the consumer throat or lungs.

A quartz banger is an attachment to a water pipe that is used as a surface to safely burn product for smoking medicinal herbal concentrate. Quartz is not a limiting feature of the banger as it may also be made of titanium, borosilicate, ceramic, and non-quartz glass. The quartz banger may be hook-shaped with a bend that may be curved not limited to 45 or 90 degrees with a sidewall that may be 2 mm thick. The banger will form an end joint for joining to the water pipe apparatus that may be 12 mm, 14 mm, 16 mm, or 18 mm wide in diameter, with male or female ends. Said quartz banger incorporates through new use color changing technology as described on the Chameleon International website, to be inserted proximal to the quartz neck area or head structure.

In accordance with a non-limiting embodiment of the invention, a quartz banger, when heated either by means of a butane torch or other heat source, will display a change in color by means of an insertion near the neck, said insertion being said licensed technology mentioned above, indicating when the quartz has reached the optimal temperature so that the consumer may remove the heat source and apply medicinal herbal concentrate to the quartz surface for consumption.

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 is a side perspective of a preferable quartz banger according to an embodiment of the present invention;

FIG. 2 is a side perspective of a preferable quartz banger according to an embodiment of the present invention.

### DETAILED DESCRIPTION OF PREFERRED EMBODIMENTS

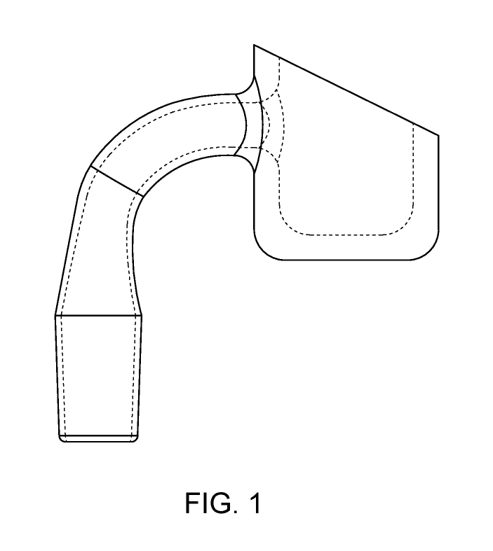

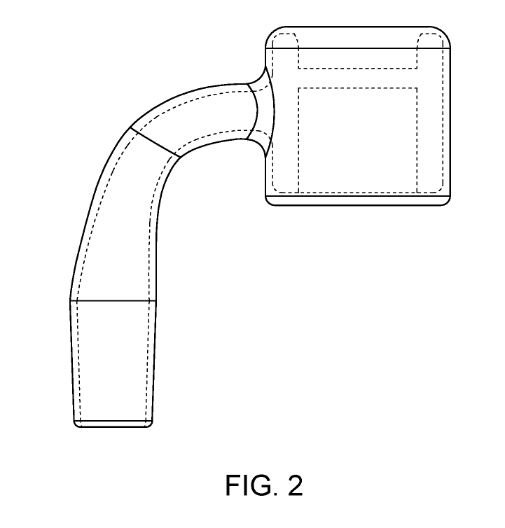

Preferred embodiments of the present invention and their advantages may be understood by referring to FIGS. 1-2, wherein like reference numerals refer to like elements.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

Headings provided herein are for convenience and are not to be taken as limiting the disclosure in any way.

The enumerated listing of items does not imply that any or all of the items are mutually exclusive, unless expressly specified otherwise.

The terms “a”, “an” and “the” mean “one or more”, unless expressly specified otherwise.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

The present invention will now be described in detail with reference to embodiments thereof as illustrated in the accompanying drawings.

FIG. 1 discloses one iteration of the quartz banger structure where the larger portion is the head area wherein one embodiment the color changing surface may be inserted; the extension from the large portion is the neck wherein one embodiment the color changing surface may be inserted, and the end of the neck is the male or female end-joint attaching to a water pipe apparatus with varying sizes of 12 mm, 14 mm, 16 mm, or 18 mm diameter.

FIG. 2 discloses another such iteration with varying head structure that allows flexibility as to the means in which concentrate may be applied to the head structure for smoking; consisting of the same neck and end-joint with like variance in diameter as other iterations.

Heat is continually applied to the bottom of the head structure, preferably by means of a butane torch, until the color changing surface is, in an embodiment, inserted near the neck until the surface is wholly changed in color, and at such indication the consumer is notified of the optimal temperature of 300-400 degrees at which concentrate may be applied to the head structure for smoking resulting in the intake of the highest amount of active ingredients, helpful medicines, and aromatics or terpenes, and with the lowest amounts of harmful combustion products.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claim

I claim:

1. A quartz banger not limited to 45 and 90 degree bends with 2 mm sidewall thickness comprising an end-joint for joining to any water pipe apparatus and said end-joint being either 12 mm, 14 mm, 16 mm, or 18 mm wide in diameter, with male or female ends; the quartz banger comprising color changing technology further indicating with specific colors when the quartz has reached the optimal temperature range of 300-400 degrees, and such color changing surface may be inserted into the quartz not limited to any area around the neck or surface composed of the head structure where product is placed.

2. The apparatus of claim 1, wherein the banger is composed of titanium.

3. The apparatus of claim 1, wherein the banger is composed of borosilicate.

4. The apparatus of claim 1, wherein the banger is composed of ceramic.

5. The apparatus of claim 1, wherein the banger is composed of non-quartz glass.
